(19) United States
(12) Patent Application Publication
(10) Pub. No.: US 2025/0082462 A1
(43) Pub. Date: Mar. 13, 2025

(54) OPHTHALMIC LENS HAVING AN EXTENDED DEPTH OF FOCUS

(71) Applicant: Alcon Inc., Fribourg (CH)

(72) Inventors: XIN HONG, FORT WORTH, TX (US); ZORAN MILANOVIC, FORT WORTH, TX (US); XIN WEI, FRISCO, TX (US)

(21) Appl. No.: 18/958,272

(22) Filed: Nov. 25, 2024

Related U.S. Application Data
(63) Continuation of application No. 17/368,962, filed on Jul. 7, 2021.
Application No. 17/368,962 is a continuation of application No. 15/947,050, filed on Apr. 6, 2018, now Pat. No. 11,083,566.
Application No. 15/947,050 is a continuation of application No. 15/055,993, filed on Feb. 29, 2016, now Pat. No. 9,968,440.

(51) Int. Cl. A61F 2/16; G02B 27/00; G02C 7/04

(52) U.S. Cl. CPC A61F 2/1613; A61F 2/1616; G02B 27/0075; G02C 7/041; A61F 2250/0053

(57) ABSTRACT

In certain embodiments, an ophthalmic lens includes an optic having an anterior surface, a posterior surface, and an optical axis. At least one of the anterior surface and the posterior surface includes a first zone extending from the optical axis to a first radial boundary and a second zone extending from the first radial boundary to the edge of the optic. The first zone includes an inner region and an outer region separated by a phase shift feature, the phase shift comprising a ridge extending outwardly from the inner region and the outer region.

### RELATED APPLICATIONS

This application is a continuation of U.S. Non-Provisional application Ser. No. 17/368,962, filed Jul. 7, 2021, which is a continuation of U.S. Non-Provisional application Ser. No. 15/947,050, filed Apr. 6, 2018 and now granted as U.S. Pat. No. 11,083,556, which is a continuation of, and claims priority to, U.S. Non-Provisional application Ser. No. 15/055,993 filed Feb. 29, 2016 and now granted as U.S. Pat. No. 9,968,440, all of which are incorporated herein in their entireties.

### FIELD

This present disclosure relates generally ophthalmic lenses and, more particularly, to ophthalmic lenses having an extended depth of focus.

### BACKGROUND

Intraocular lenses (IOLs) are routinely implanted in patients' eyes during cataract surgery to replace the natural crystalline lens. The optical power of the natural crystalline lens can vary under the influence of the ciliary muscles to provide accommodation for viewing objects at different distances from the eye. Many IOLs, however, provide a monofocal power with no provision for accommodation. Multifocal IOLs are also known that provide a distance optical power as well as a near optical power (e.g., by employing diffractive structures), thereby providing a degree of pseudoaccommodation. There is, however, still a need for improved IOLs that can provide pseudo-accommodative optical power.

### SUMMARY

The present disclosure generally concerns ophthalmic lenses (e.g., IOLs) that provide (1) controlled variation of multiple phase shifts within the pupil region to extend the depth-of-focus and (2) power adjustment in the central sub-area of the pupil region to shift the through-focus curve and to rebalance the energy between intermediate correction and the distance correction. In certain embodiments, an ophthalmic lens includes an optic having an anterior surface, a posterior surface, and an optical axis. At least one of the anterior surface and the posterior surface includes a first zone extending from the optical axis to a first radial boundary and a second zone extending from the first radial boundary to the edge of the optic. The first zone includes an inner region and an outer region separated by a phase shift feature, the phase shift comprising a ridge extending outwardly from the inner region and the outer region.

### BRIEF DESCRIPTION OF THE DRAWINGS

For a more complete understanding of the present disclosure and the advantages thereof, reference is now made to the following description taken in conjunction with the accompanying drawings in which like reference numerals indicate like features and wherein:

FIGS. 1A-1B illustrate and example embodiment of an intraocular lens having an extended depth of focus, according to certain embodiments of the present disclosure;

FIG. 2 illustrates an plot of surface sag versus radial distance from the optical axis for an exemplary optic having inner and outer zones with the same base curvature, according to certain embodiments of the present disclosure;

FIG. 3 illustrates a through focus plot for the optic surface profile depicted in FIG. 2 as compared to the through focus plot for a standard aspheric optic, according to certain embodiments of the present disclosure;

FIG. 4 illustrates a plot of surface sag versus radial distance from the optical axis for an exemplary optic having inner and outer zones with different base curvatures, according to certain embodiments of the present disclosure; and

FIG. 5 illustrates a through focus plot for the optic surface profile depicted in FIG. 4 as compared to the through focus plot for the optic depicted in FIG. 2, according to certain embodiments of the present disclosure.

The skilled person in the art will understand that the drawings, described below, are for illustration purposes only. The drawings are not intended to limit the scope of the applicant's disclosure in any way.

### DETAILED DESCRIPTION

The present disclosure is generally directed to an ophthalmic lens (such as an IOL) having a surface profile that produces a controlled variation of phase shifts in light waves passing through various regions of the lens in a manner that extends the depth-of-focus. In the following description, the lens features providing an extended depth of focus are described in connection with intraocular lenses (IOLs). However, the present disclosure contemplates that those features can also be applied to other ophthalmic lenses, such as contact lenses. As used herein, the term intraocular lens (and its abbreviation IOL) are used to describe lenses that are implanted into the interior of the eye to either replace the eye's natural lens or to otherwise augment vision regardless of whether or not the natural lens is removed.

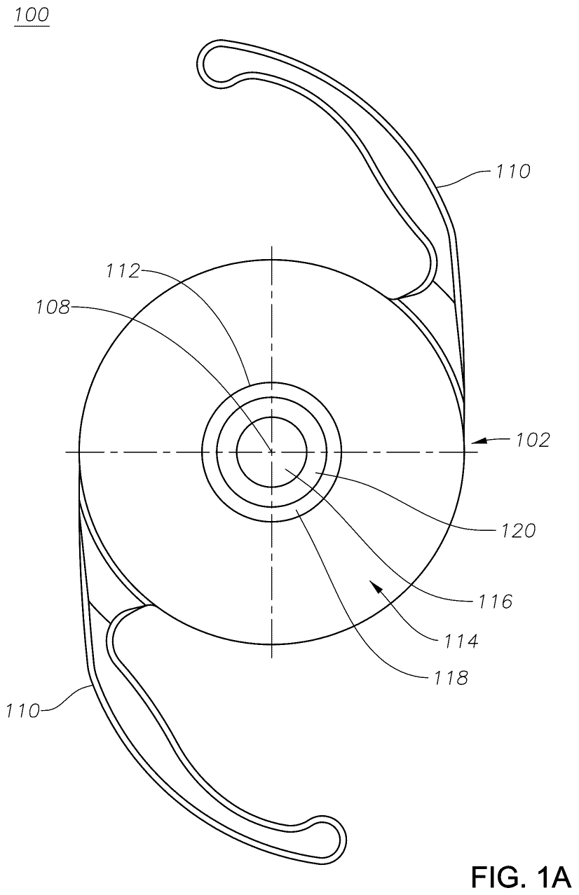

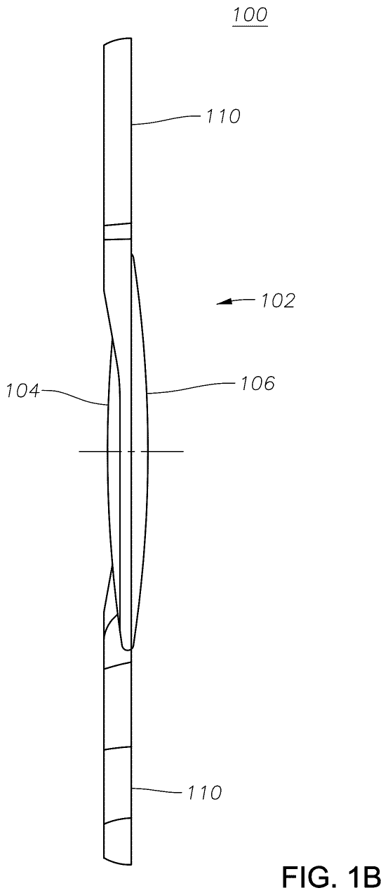

FIGS. 1A-1B illustrate and example embodiment of an intraocular lens 100 having an extended depth of focus, according to certain embodiments of the present disclosure. IOL 100 includes an optic 102 having an anterior surface 104 and a posterior surface 106 that are disposed about an optical axis OA 108. IOL 100 may further include a plurality of haptics 110 generally operable to position and stabilize IOL 100 within the capsular bag of a patient's eye.

As shown in FIG. 1A, the anterior surface 104 of optic 102 includes a first zone 112 extending from the optical axis 108 to a first radial boundary and a second zone 114 extending from the first radial boundary to the edge of the optic 102. Additionally, the first zone 112 may include an inner region 116 and an outer region 118 separated by a phase shift feature 120. In general, the above-described surface features of optic 102 may produce varying amount of phase shift of light waves passing through optic 102 (depending upon the region of the optic 102 the light waves pass through), and constructive interference between the light waves having varying amounts of phase shift may produce an extended depth of focus. Although the above-described first and second zones 112, 114 are depicted and described as being located on anterior surface 104 of optic 102, the present disclosure contemplates that first and second zones 112, 114 may additionally or alternatively be located on posterior surface 106 of optic 102.

In certain embodiments, phase shift feature 120 may include a ridge projecting anteriorly from the anterior surface 104 of optic 102. As a result, moving radially outward from the optical axis 108, phase shift feature 120 may result in two phase shift steps. For example, the surface profile of the first zone may be defined by the following equation:

$${Z}_{first zone} = {Z}_{b a s e} + {Z}_{2 p s} \qquad Eq . ( 1 )$$

In Eq. (1), Z_{base} may define a base sag profile for the first zone according to the following equation:

$${Z}_{b a s e} = \frac{c {r}^{2}}{1 + \sqrt{1 - ( 1 + k ) {c}^{2} {r}^{2}}} + {a}_{2} {r}^{2} + {a}_{4} {r}^{4} + {a}_{6} {r}^{6} + … + {a}_{n} {r}^{n} \qquad Eq . ( 2 )$$

wherein,
r is a radial distance from the optical axis 108;
c is a base curvature of the first zone 112;
k is a conic constant; and
a₂, a₄, a₆. . . , and aₙ are, respectively, second, fourth, sixth, . . . , and n^{th} order coefficients.

In certain embodiments, the equation defining Z_{base} may only include second, fourth, and sixth order coefficients. In other words, Z_{base} may define a base sag profile for the first zone according to the following equation:

$${Z}_{b a s e} = \frac{c {r}^{2}}{1 + \sqrt{1 - ( 1 + k ) {c}^{2} {r}^{2}}} + {a}_{2} {r}^{2} + {a}_{4} {r}^{4} + {a}_{6} {r}^{6} \qquad Eq . ( 3 )$$

Although Eq. (2) and Eq. (3) generally define aspheric surface profiles, the present disclosure contemplates that the constants includes in those equations may be selected such that they define a spheric profile. In other words, the base curvature of the first zone (Z_{base}) may be either spheric or aspheric.

In Eq. (1), Z₂ₚₛ may be added to the base sag profile (Z_{base}) and may, in part, define the features of the phase shift region 120. For example, Z₂ₚₛ may be defined by the following equation:

$${Z}_{2 p s} = \{ \begin{matrix}0 & {r}_{0} \leq r < {r}_{1} \\ ( r - {r}_{1} ) / ( {r}_{2} - {r}_{1} ) * {Δ}_{1} & {r}_{1} \leq r < {r}_{2} \\ {Δ}_{1} & {r}_{2} \leq r < {r}_{3} \\ {Δ}_{1} + ( r - {r}_{3} ) / ( {r}_{4} - {r}_{3} ) * {Δ}_{2} & {r}_{3} \leq r < {r}_{4} \\ {Δ}_{1} + {Δ}_{2} & {r}_{4} \leq r < {r}_{5}\end{matrix} ; \qquad Eq . ( 4 )$$

where,
r is a radial distance from the optical axis 108;
r₀ is the optical axis 108;
the inner region 116 extends from the optical axis 108 to r₁
the phase shift feature 120 extends from r₁ to r₄;
the outer region 118 extends from r₄ to r₅;
Δ₁ is a step height of the phase shift 120 feature relative to the inner region 116; and
Δ₂ is a step height of the phase shift feature relative to the outer region 118.

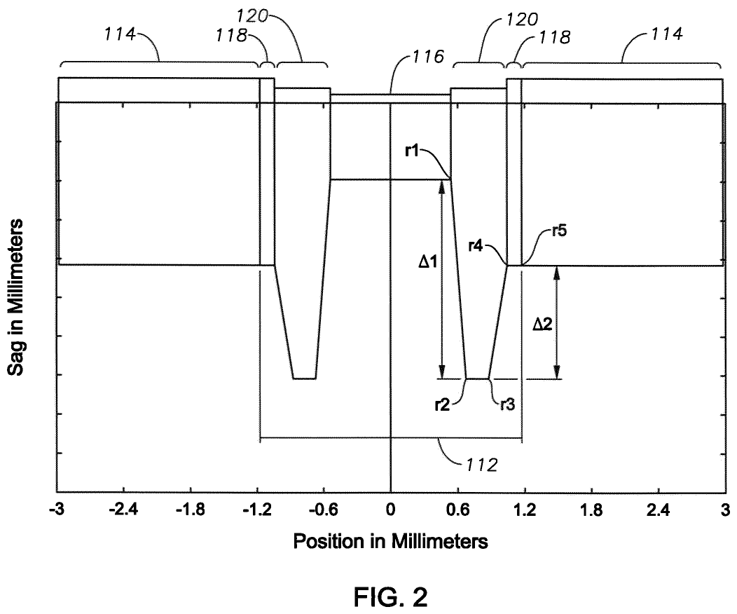

The overall surface profile of optic 102, as defined by Eqs. (1)-(4), may be graphically represented as a plot of sag vs. radial distance from the optical axis 108, as shown in FIG. 2. In the plot of FIG. 2, the sag values have been normalized by removing the contribution of Z_{base} (i.e., the plotted sag value corresponds only to Z₂ₚₛ). Additionally, in the plot of FIG. 2, the sag profile is constant for the first zone 112 and the second zone 114. In other words, it is assumed that Eq. (1) defines the surface profile of the entire optic 102 as opposed to only the first zone 112 (meaning that, in Eq. (4), r₅ corresponds to the radius of the entire optic 102).

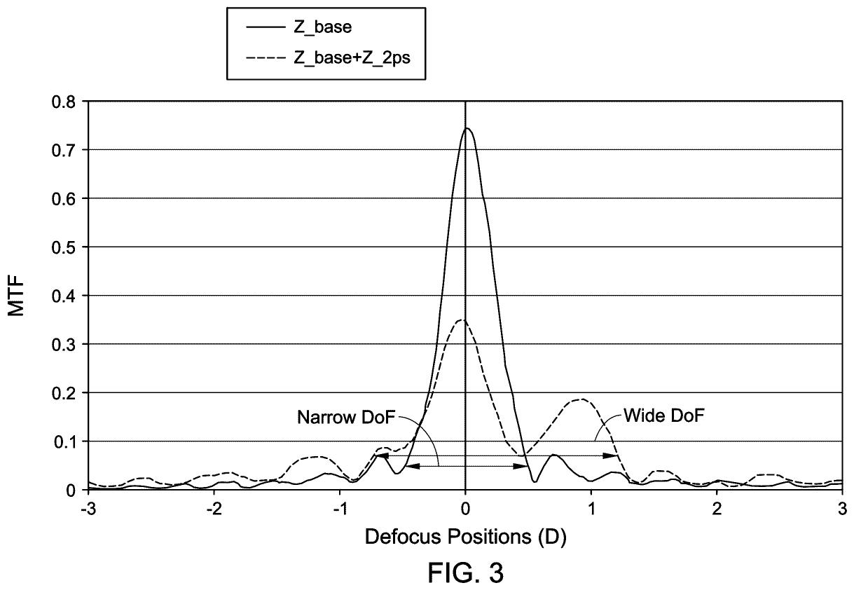

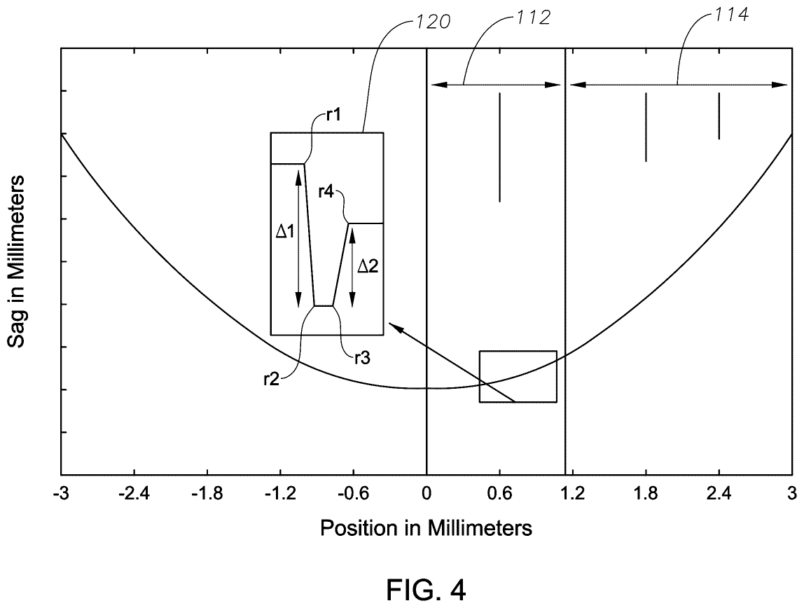

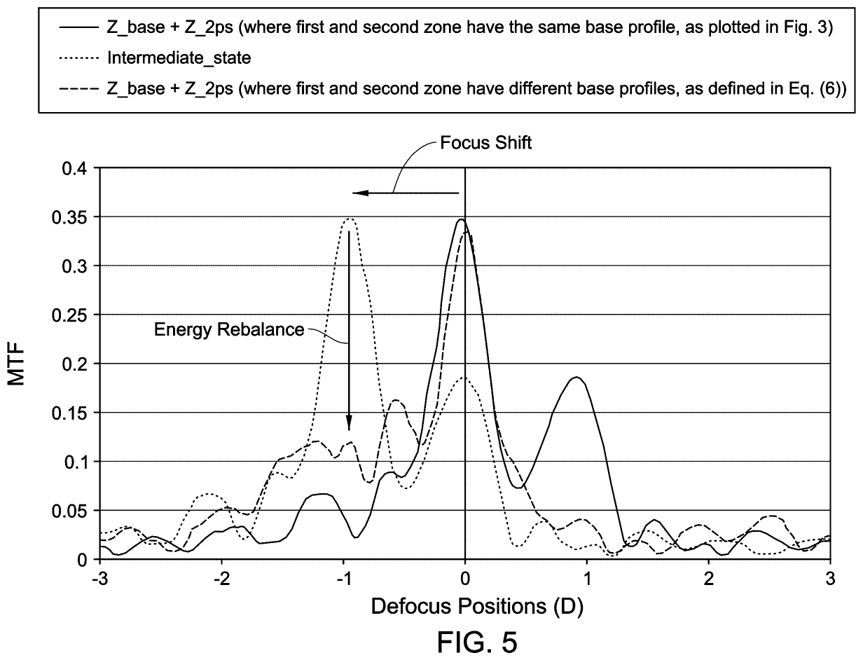

FIG. 3 illustrates a through focus plot for the optic surface profile depicted in FIG. 2 as compared to the through focus plot for a standard aspheric optic (i.e., an optic having a surface profile defined only by Eq. (3) (Z_{base}) without the addition of Eq. (4) (Z₂ₚₛ)), according to certain embodiments of the present disclosure. As is illustrated, the addition of the surface profile depicted in FIG. 2 (including the phase shift feature 120 represented by Z₂ₚₛ) results in a wider depth of focus as compared to a standard aspheric lens.

In certain embodiments, the base sag profile may be different for the first zone 112 and the second zone 114. For example, the surface profile of the optic 102 may be defined by the following equation:

$${Z}_{optic} = {Z}_{base} + {Z}_{2 p s} \qquad Eq . ( 5 )$$

$$where ,$$

$${Z}_{base} = \{ \begin{matrix}\frac{c {r}^{2}}{1 + \sqrt{1 - ( 1 + k ) {c}^{2} {r}^{2}}} + {a}_{2} {r}^{2} + {a}_{4} {r}^{4} + {a}_{6} {r}^{6} & {r}_{0} \leq r < {r}_{5} \\ \frac{{c}^{′} {r}^{2}}{1 + \sqrt{1 - ( 1 + {k}^{′} ) {c}^{′2} {r}^{2}}} + {a}_{2}^{′ {r}^{2}} + {a}_{4}^{′ {r}^{4}} + {a}_{6}^{′} {r}^{6} & {r}_{5} \leq r < {r}_{6}\end{matrix} \qquad Eq . ( 6 )$$

$${Z}_{2 p s} = \{ \begin{matrix}0 & 0 \leq r < {r}_{1} \\ ( r - {r}_{1} ) / ( {r}_{2} - {r}_{1} ) * {Δ}_{1} & {r}_{1} \leq r < {r}_{2} \\ {Δ}_{1} & {r}_{2} \leq r < {r}_{3} \\ {Δ}_{1} + ( r - {r}_{3} ) / ( {r}_{4} - {r}_{3} ) * {Δ}_{2} & {r}_{3} \leq r < {r}_{4} \\ {Δ}_{1} + {Δ}_{2} & {r}_{4} \leq r < {r}_{5} \\ {Δ}_{3} & {r}_{5} \leq r < {r}_{6}\end{matrix} \qquad Eq . ( 7 )$$

$${Δ}_{3} = [ \frac{( {cr}_{5}^{2} )}{( 1 + \sqrt{( 1 - ( 1 + k ) {c}^{2} {r}_{5}^{2} )} )} + {a}_{4} {r}_{5}^{4} + {a}_{6} {r}_{5}^{6} + {Δ}_{1} + {Δ}_{2} ] - [ \frac{( {c}^{′} {r}_{5}^{2} )}{( 1 + \sqrt{( 1 - ( 1 + {k}^{′} ) {c}^{′2} {r}_{5}^{2} )} )} + {a}_{4}^{′} {r}_{5}^{4} + {a}_{6}^{′} {r}_{5}^{6} ] \qquad Eq . ( 8 )$$

r is a radial distance from the optical axis 108;
r₀ is the optical axis 108;
the first zone 112 extends from optical axis 108 to r₅, with the inner region 116 extending from the optical axis 108 to r₁, the phase shift feature 120 extending from r₁ to r₄, and the outer region 118 extending from r₄ to r₅;
the second zone 114 extends from r₅ to r₆;
c is a base curvature of the first zone 112;
k is a conic constant of the first zone 112; and
a₂, a₄, and a₅ are, respectively, second, fourth, and sixth order coefficients of the first zone 112;
c′ is a base curvature of the second zone 114;
k′ is a conic constant of the second zone 114; and
a₂′, a₄′, and a₆′ are, respectively, second, fourth, and sixth order coefficients of the second zone 114;
Δ₁ is a step height of the phase shift feature 120 relative to the inner region 116; and
Δ₂ is a step height of the phase shift feature 120 relative to the outer region 118.

Although base profiles defined in Eq. (6) above only include second, fourth, and sixth order coefficients, the present disclosure contemplates that those base profiles could alternatively be defined as including any suitable number of higher order coefficients (as in Eq. (1)).

Because the first zone 112 and the second zone 114 have different base sag profiles, Δ₃ (as defined in Eq. (8)) may provide for a smooth transition between the first zone 112 and the second zone 112. For example, the first zone 112 may be modified with a different base curvature (c), conic constant (k), and/or higher order coefficients (a₂, a₄, a₆) as compared to second zone 114 in order to shift the through focus curve in the myopic direction as compared to the through focus curve depicted in FIG. 3. FIG. 4 illustrates a plot of surface sag versus radial distance from the optical axis for an optic 102 having a surface profile defined by Eqs. (5) through (8), according to certain embodiments of the present disclosure. The surface profile plotted in FIG. 4 assumes the following values:

TABLE 1

| | |
|---|---|
| r₁ (mm) | 0.55 |
| r₂ (mm) | 0.65 |
| r₃ (mm) | 0.87 |
| r₄ (mm) | 1.05 |
| r₅ (mm) | 1.11 |
| r₆ (mm) | 3.00 |
| Δ₁ (μm) | −1.02 |
| Δ₂ (μm) | 0.59 |
| c (1/mm) | 19.05 |
| k | 5.99 |
| a₂ (1/mm) | 0 |
| a₄ (1/mm³) | 0 |
| a₆ (1/mm⁵) | 0 |
| c′ (1/mm) | 20.74 |
| k′ | −43.56 |
| a₂′ (1/mm) | 0 |
| a₄′ (1/mm³) | 0.00019 |
| a₆′ (1/mm⁵) | −0.00002 |

The values listed in Table 1 are provided for exemplary purposes only and the present disclosure contemplates that each of the values may have a range of different values. As examples, the present disclosure contemplates that r₁ may fall in the range of 0.3 mm to 0.7 mm, r₄ may fall in the range of 0.8 mm to 1.2 mm, the distance between r₁ and r₂ may fall in the range of 0 mm to 0.2 mm, and the distance between r₃ and r₄ may fall in the range of 0 mm to 0.2 mm. As additional examples, the present disclosure contemplates that Δ₁ may fall within the range of −1.5 μm and −0.5 μm and Δ₂ may fall within the range of 0.3 μm and 0.9 μm.

FIG. 5 illustrates a through focus plot for the optic surface profile depicted in FIG. 4 as compared to the through focus plot for the optic depicted in FIG. 2, according to certain embodiments of the present disclosure. As discussed above, modifying the first zone 112 with a different base curvature, conic constant, and/or higher order coefficients (1) rebalances the energy between intermediate and distance correction, and (2) shifts the through focus curve in the myopic direction (near target direction) as compared to the through focus curve for an optic in which the first zone 112 and the second zone 114 have the same base curvature.

A variety of techniques and materials can be employed to fabricate the above-described IOLs 100. For example, the optic 102 of an IOL 100 can be formed of a variety of biocompatible polymeric materials. Some suitable biocompatible materials include, without limitation, soft acrylic polymers, hydrogel, polymethymethacrylate, polysulfone, polystyrene, cellulose, acetate butyrate, or other biocompatible materials. By way of example, in one embodiment, the optic 102 may be formed of a soft acrylic polymer (cross-linked copolymer of 2-phenylethyl acrylate and 2-phenylethyl methacrylate) commonly known as Acrysof. The haptics 104 of the IOLs 100 can also be formed of suitable biocompatible materials, such as those discussed above. While in some cases, the optic 102 and haptics 104 of an IOL can be fabricated as an integral unit, in other cases they can be formed separately and joined together utilizing techniques known in the art.

It will be appreciated that various of the above-disclosed and other features and functions, or alternatives thereof, may be desirably combined into many other different systems or applications. It will also be appreciated that various presently unforeseen or unanticipated alternatives, modifications, variations or improvements therein may be subsequently made by those skilled in the art which alternatives, variations and improvements are also intended to be encompassed by the following claims.

1-20. (canceled)

21. An ophthalmic lens, comprising
an optic comprising an optical axis and an optical profile extending radially from the optical axis, the optical profile comprising:
a first zone having a first aspheric profile,
a second zone having a second aspheric profile different from the first aspheric profile, and
a phase shift feature;
wherein the phase shift feature is configured to produce varying amounts of phase shift of light waves passing through the optic such that constructive interference between the light waves having varying amounts of phase shift provides an extended depth of focus.

22. The ophthalmic lens of claim 21, wherein the phase shift feature comprises an annular ring centered about the optical axis, the annular ring having an outer diameter between 1.6 mm and 2.4 mm.

23. The ophthalmic lens of claim 22, wherein the annular ring has a width between 0.1 mm and 1 mm.

24. The ophthalmic lens of claim 21, wherein the phase shift feature comprises a ridge projecting anteriorly from an anterior surface of the optic.

25. The ophthalmic lens of claim 21, wherein the phase shift feature comprises a ridge projecting posteriorly from a posterior surface of the optic.

26. The ophthalmic lens of claim 21, wherein the phase shift feature comprises:
a first phase shift step;
a second phase shift step; and
a region of increased thickness extending from the first phase shift step to the second phase shift step.

27. The ophthalmic lens of claim 26, wherein:
the first phase shift step has a first step height;
the second phase shift step has a second step height; and
the second step height is greater than the first step height.

28. The ophthalmic lens of claim 21, wherein the phase shift feature is positioned within the first zone.

29. The ophthalmic lens of claim 21, wherein the ophthalmic lens comprises an intraocular lens.

30. The ophthalmic lens of claim 21, wherein the ophthalmic lens comprises a contact lens.

31. An ophthalmic lens, comprising:
an optic comprising a surface and an optical axis, the surface comprising:
a first zone extending from the optical axis to a first radial boundary, the first zone having a first base curvature;
a second zone extending from the first radial boundary towards the edge of the optic, the second zone having a second base curvature; and
a phase shift feature within the first zone configured to produce varying amounts of phase shift of light waves through the optic;
wherein the first base curvature of the first zone is configured to provide increased range of focus in the myopic direction.

32. The ophthalmic lens of claim 31, wherein the first base curvature comprises a first aspheric profile, and the second base curvature comprises a second aspheric profile.

33. The ophthalmic lens of claim 32, wherein the first base curvature and the second base curvature have different aspheric surface profiles.

34. An ophthalmic lens, comprising:
an optic comprising an optical axis and an optical profile extending radially from the optical axis, the optical profile comprising:
a first region extending radially from the optical axis toward a first boundary,
a second region extending radially from a second boundary towards the edge of the optic, and
a phase shift feature positioned between the first boundary and the second boundary, the phase shift feature comprising an annular ring centered about the optical axis, wherein the annular ring has an outer diameter between 1.6 mm and 2.4 mm.

35. The ophthalmic lens of claim 34, wherein the annular ring has a width between 0.1 mm and 1 mm.

36. The ophthalmic lens of claim 34, wherein the annular ring has a maximum step height greater than 1 μm.

37. An ophthalmic lens, comprising:
an optic comprising an anterior surface, a posterior surface, and an optical axis;
wherein at least one of the anterior surface and the posterior surface comprises:
a phase shift feature comprising at least one phase shift step;
a first zone extending radially from the optical axis toward an inward boundary of a phase shift feature, the first zone comprising a first curvature configured to produce a first phase shift of light waves passing through the first zone; and
a second zone extending radially from an outward boundary of the phase shift feature toward the edge of the optic, the second zone having a second curvature that is different than the first curvature, the second curvature configured to produce a second phase shift of light waves passing through the second zone, the second phase shift being different than the first phase shift;
wherein the phase shift step of the phase shift feature is configured to produce a third phase shift of light waves passing through the phase shift feature, the third phase shift being different than the first phase shift and the second phase shift; and
wherein the first phase shift, the second phase shift, and the third phase shift are configured to produce constructive interference such that a depth of focus of the optic is extended in a myopic direction to provide increased near vision.
